(19) United States
(12) Patent Application Publication
(10) Pub. No.: US 2007/0110784 A1
(43) Pub. Date: May 17, 2007

(54) Thermally reversible implant

(75) Inventors: Yu-Ling Cheng, Mississauga (CA); Michael H. May, Brantford (CA); John L. Semple, Toronto (CA); Hai-Hui Lin, Mason, OH (US)

Correspondence Address: BORDEN LADNER GERVAIS LLP, 1100-100 QUEEN ST, OTTAWA, ON, K1P 1J9, CA

(73) Assignee: RIMON THERAPEUTICS LTD., TORONTO (CA)

(21) Appl. No.: 10/555,455

(22) PCT Filed: May 3, 2004
(86) PCT No.: PCT/CA04/00670

§ 371 Date: Jan. 12, 2007

(30) Foreign Application Priority Data
May 2, 2003 (US) 10/428520

(51) Int. Cl. A61F 2/02; A61K 8/73; A61K 8/81

(52) U.S. Cl. 424/423; 424/70.13; 424/70.16

(57) ABSTRACT

The invention relates to the use of a thermal reversible gel, such as a copolymer composition, as a biological filler or implant. The gel has a semi-solid form at body temperature, but upon cooling to a temperature below a threshold level, the gel is liquefied and can be re-shaped, re-sized, manipulated or removed from the body. The gel may be used as a subcutaneous implant, a biological filler, joint or tissue spacer, for wrinkle filling or other cosmetic implants, as a soft-tissue replacement for reconstructive surgery, or as a barrier within the lumen of a biological structure, such as a blood vessel. The implant may be used to provide reversible birth control by providing, for example, a reversible barrier to the cervix or a reversible blockage of the lumen of the vas deferens.

### FIELD OF THE INVENTION

The present invention relates generally to thermally reversible polymer implants for use in biological applications.

### BACKGROUND OF THE INVENTION

Prior art implants for use in biological applications generally do not allow thermally reversible removal or modification of the substance used. For example, the use of silicone implants and polymeric implants do not allow easy modification of shape, volume or placement in a reversible way, once the implant is in place.

In reconstructive and cosmetic surgery and other cosmetic procedures, the success or failure of the procedure depends in part on the satisfaction of the patient with the appearance of their altered physical attribute. There age very few methods available, short of a subsequent surgery or repeat procedures, to correct errors or affect changes to a cosmetic alteration.

With an aging population and a concurrent emphasis on youthful appearance, a number of methods have arisen for reducing facial lines and wrinkles. One such method involves injection of a toxin below the skin to cause a localized immune reaction that smoothes out wrinkles. One problem with this method is the potential or perceived danger to the patient due to unexpected reactions to the toxin, Other methods involve injection of natural materials (e.g., collagen and hyaluronic acid) under the wrinkle to raise the skin. One problem with these implants is the potential or perceived danger that these materials may be immunogenic, be allergenic or carry animal-bone diseases (e.g., mad cow disease or its human equivalent—Creutzfeldt-Jacob Disease), In addition, these implants begin to degrade upon implantation, making it difficult or impossible to remove them, if necessary. In some cases, small, non-degradable beads (e.g., polymethymethacrylate) are suspended in wrinkle fillers to give them a longer-lasting effect. These small beads become surrounded by fibrous tissue as part of the normal foreign body reaction to implants, which prolongs their effect, but makes them impossible to remove, if desired.

Current methods of birth control are either irreversible, or only reversible through lengthy surgical procedures (for example, a reverse vasectomy). Other methods, such as “the pill” use pharmaceutical means to cause a temporarily infertile state. Subject compliance is necessary for the success of such methods. There is a need for reversible long-term options for birth control for both men and women.

Block and graft copolymers are used for a variety of physiological and industrial applications. The solubility of a copolymer in a particular solvent depends inter alia on the characteristics of the monomeric components incorporated into the copolymer.

Polymers capable of gelation induced by environment changes are known. Solvent-induced gelation has also been exploited as a mechanism for producing in situ gelable materials. The solvent-induced gelation concept employs a polymer that is soluble in a non-aqueous solvent, but insoluble in water. When a non-aqueous solution of such a polymer is injected into an aqueous environment, the non-aqueous solvent is exchanged for water and the polymer precipitates, forming a solid mass in situ. Solvent-induced gelation systems have the disadvantage that the initial fluid form of the polymer is formed in a solvent other than the solvent in which the gel eventually forms. U.S. Pat. No. 5,744,153 (Apr. 28, 1998) and No. 5,759,563 (Jun. 2, 1998), both to Yewey et al., describe a composition for in situ formation of a controlled drug release implant based on the solvent-induced gelation concept.

A series of patents to Dunn et. al. also describe a solvent-induced gel composition (U.S. Pat. No. 5,739,176 issued Apr. 14, 1998; No. 5,733,950 issued Mar. 31, 1998; No. 5,340,849 issued Aug. 23, 1994; U.S. Pat. Nos. 5,278,201 and 5,278,204 both issued Jan. 11, 1994; and U.S. Pat. No. 4,938,763 issued Jul. 3, 1990). The composition includes a water-insoluble polymer and a drug solubilized in an organic solvent carrier. When the composition is injected into a physiological (aqueous) environment, such as a human subject, the polymer precipitates to form a solid mass. Solvent-induced gel compositions have the disadvantage that an organic solvent is injected into a subject merely to carry the polymer and drug in a liquid form. Thus, the organic solvent must subsequently be metabolized or cleared by the body.

Self-assembling hydrogels have been receiving easing attention in the last few years, both for their intrinsic scientific interest, and for their potential clinical ad non-clinical applications. A number of elegant mechanisms for self-assembling hydrogels have been proposed. Nagahara et al. showed that gels can be formed by complexation between complementary oligonucleotides grafted onto hydrophilic polymers (Polymer Gels and Networks, 4:(2) 111-127, 1996). Miyata et al. prepared antigen sensitive hydrogels based on antigen-antibody banding (Miyata et al., Macromolecules, 32: (6) 2082-2084, 1999; Miyata, Nature, 399: (6738) 766-769, 1999). Petka et al. illustrated a gelation mechanism using triblock copolymers containing a central hydrophilic core and terminal leucine zipper peptide domains (Science, 281: (5375)389-392, 1998). The terminal domains form coil-coil dimers or higher order aggregates to provide crosslinking when cooled from above its pH-dependent melting point. Thermoreversibility was demonstrated with some hysteresis due to the slow kinetics of coil-coil interactions.

Triblock copolymers having a central hydrophobic polypropylene oxide) (PPO) segment and hydrophilic poly(ethylene oxide) (PEO) segments attached at each end are commercially available. The aqueous solution of these triblock copolymers (PEO-PPO-PEO) have a fluid consistency at room temperature, and turn into weak gels when warmed to body temperature by forming oil-in-water micelles in aqueous solution. The gelation of the polymer is believed to occur via the aggregation of the micelles (Cabana et al., J. Coll. Int. Sci., 190(1997) 307).

A group led by S. W. Kim have reported the development of thermosensitive biodegradable hydrogels (Jeong et al., J. Controlled Release, 62 (1999) 109-114; Jeong et al., Macromolecules, 32: (21) 7064-7069, 1999; Jeong et al., Nature, 388 (1997) 860-862). These hydrogels are block copolymers of PEO and poly(L-lactic acid) (PLLA) in either a di-block architecture PEO-PLLA, or a tri-block architecture PEO-PLLA-PEO. They also report triblock copolymers of poly(ethylene oxide) and poly(lactide-co-glycohde) (PLGA) having the architecture PEO-PLGA-PEO. Aqueous solutions of these polymers were reported to undergo temperature-sensitive phase transitions between fluid solution and gel phases. In aqueous solution, these polymers form micelles composed of hydrophobic cores (either PLGA or PLLA) and hydrophilic surfaces (PEO). Gelation is believed to be due to the aggregation of micelles driven by hydrophobic interactions. This group has also discussed the synthesis of PEO copolymers in multi-armed star shaped architectures having polycaprolactone (PCL) or PLLA chains attached to the PEO arms.

Another class of in situ gelable materials is based on polymers made from proteins, or “protein polymers”, Cappello, et al. (J Controlled Release 53 (1998) 105-117) reported gel-forming block copolymers based on repeating amino acid sequences from silk and elastin proteins. When heated to body temperature, the proteins self-assemble via a hydrogen bond mediated chain crystallization mechanism to form an irreversible gel. The gelation occurs over a relatively long time period of more than 25 minutes.

Although a variety of gelling or precipitatable polyethylene glycol/poly(N-isopropylacrylamide) copolymers have been synthesized, none was designed and synthesized with in situ gelation applications in mind. See, for example Yoshioka et al., J.M.S Pure Appl. Chem. A31: (1) 109-112, 1994; Yoshioka, J.M.S. Pure Appl. Chem., A31: (1) 113-120, 1994; Yoshioka, J.M.S Pure Appl. Chem., A31; (1) 121-125, 1994; Kaneko, Macromolecules, 31: 6099-6105, 1998; Topp, et al., Macromolecules, 30: 8518-8520, 1997; and Virtanen, Macromolecules, 33: 336-341, 2000.

Topp et al. disclose block copolymers of PEG and PNIPAAm having the structure of either PNIPAAm-PEG or PNIPAAm-PEG-PNIPAAM which form spherical micelles in aqueous solution (Macromolecules, 30: 8518-8520, 1997). The block copolymers were synthesized by the Ce⁺⁴ initiated attachment of NIPAAm monomers onto the hydroxyl terminals of PEG chains. It was shown that as PNIPAAm segments grew in length during synthesis, micelles having a PNIPAAm core and PEG corona were formed, and the polymerization of PNIPAAm chains continued in the core of the micelles. The copolymers formed by Topp et al. are of a form appropriate for use in a surfactant compositon for drug loaded micelles. However, micelles are isolated entities having no load bearing characteristics, do not form gels, and the formation of micelles is associated with a dilute solution state.

The block copolymer formed by Topp et al. consisted of compositions with PNIPAAm to PEG mass ratios (M_{n/PNIPAAm}/M_{n,PEG}) raging from about 0.14 to 0.48, and they found that block copolymers with a M_{n,PNIPAAm}/M_{n,PEG} ratio exceeding ⅓ show aggregation in water at temperatures below the lower critical solution temperature (LCST) at which a solubility change occurs, and thus are less useful for micelle formation than copolymers with ratios less than ⅓.

There is a need for a gelable polymer co-position capable of thermally reversibly forming a strong gel in situ.

### SUMMARY OF THE INVENTION

It is an object of the present invention to provide a biological implant that is thermally reversible so that it may be cooled for easer removal from the site of implantation.

The invention provides a thermally reversible biological implant comprising a copolymer and an aqueous solvent, the copolymer having the structure A(B)n, wherein: n is an integer greater than 0; A is soluble in the solvent; B is convertible from soluble to insoluble in the solvent as a function of temperature; and the implant is convertible from liquid to gel between 5 and 37° C.

Further, the invention provides a method of forming a removable implant in an animal comprising inserting thermally reversible gel into said animal, said gel having a semi-solid form at body temperature and a liquid form upon cooling to a temperature below a threshold temperature, said threshold temperature preferably being at least 5° C. below body temperature.

Additionally, the invention a method of forming a removable plant, as described herein, in an animal, comprising the steps of (i) forming a gelable composition comprising the copolymer and the solvent, and (ii) inserting said composition into a subject to form an in situ implant or heating said composition to at least said gelling temperature to form an in vitro implant.

In one aspect, the invention provides a process for preparing a thermally reversible gel by reacting PEG and NiPAAm in in the presence of ceric ammonium nitrate.

Additionally, the invention provides methods for modifying the gelation of the gelable composition, as well its properties in the liquid and gel states.

Other aspects and features of the present invention will become apparent to those skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIG. 1 is a schematic diagram of block copolymer architectures AB, A(B)₂, A(B)₄ and A(B)₈ and graft copolymer architectures A(B)₂ and A(B)₃, according to one aspect of the invention.

FIG. 2 is a schematic diagram of copolymer architectures A(CB)₂ and A(CB)₄ according to one aspect of the invention.

FIG. 3 illustrates an A(B)₄ polymer of PEG and PNIPAAm in aqueous solution. Picture A illustrates a 20% wt A(B)₄ solution at 25° C., while picture B illustrates a 20% wt A(B)₄ gel at 37° C.

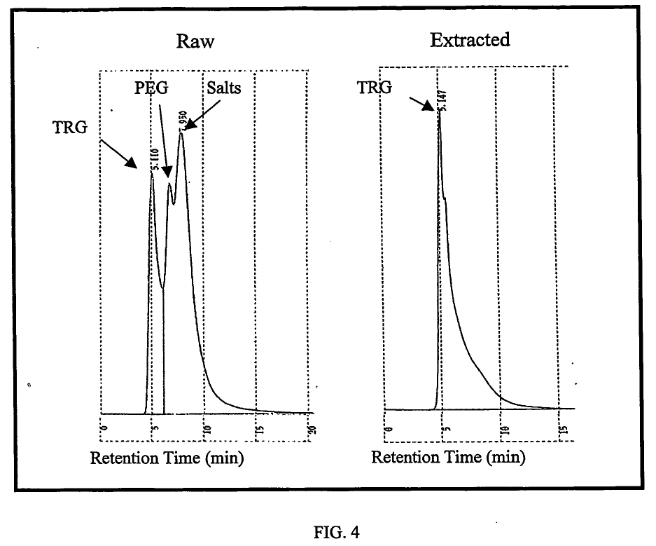

FIG. 4 illustrates gel permeation chromatograms of raw and extracted thermoreversible gel (TRG) according to an embodiment of the invention described in Example 2.

FIG. 5 illustrates TRG solution viscosity as a function of concentration (measured at 20° C. and 1000 s⁻¹).

FIG. 6 illustrates the effect of TRG concentration on the rheological measurements G′, G″ and stress at break. FIG. 7 illustrates the effect of TRG solvent osmolarity on gelation temperature.

FIG. 8 illustrates the modulation of liquid loss subsequent to TRG gelation by incorporation of additives (polyethylene glycol, mol. wt. 1,000,000 and carboxymethylcellulose, low viscosity) to the solvent.

### DETAILED DESCRIPTION

The invention provides a thermally reversible biological implant comprising a copolymer and an aqueous solvent. The copolymer has a structure A(B)n, wherein n is greater than 0, A is soluble in the solvent, and B is convertible from soluble to insoluble in the solvent as a function of temperature. The implant is convertible from liquid to gel between 5 and 37° C.

The specification sometimes makes use to a composition. Generally, use of the word “composition” refers to the mixture comprising the copolymer and the solvent.

The implant can be used as a wrinkle filler, a tissue expander, a joint spacer, a tissue spacer, a vessel blocker, a cosmetic enhancer, or a breast implant filler, among a variety of other uses.

As a wrinkle filler, the implant can be injected or otherwise placed subcutaneously in a liquid form and the body temperature allows gelling to occur. In this way, the filler advantageously can be shaped or spread thinly to achieve the desired effect while still in a liquid form. Similarly, for cosmetic or reconstructive surgery applications, the filler can be applied to a selected area of the body in a liquid form (or can be formed prior to insertion as described herein), and can be manipulated into the desired shape or to fill a desired volume. The invention has the advantage that if a subject is not satisfied with the results of the application, the effect can be changed and manipulated by application of cold directly to the region of the implant, provided that the threshold temperature is achieved by the implant. Reconstructive surgery or aesthetic enhancement may incorporate the filler or implant of the invention. Regions of the face, such as cheeks, nose, eyes, and ears (soft tissue) can be reconstructively augmented or enhanced using the invention.

As a joint spacer, the thermally reversible filler can be used to keep the components of joints spaced apart, such as in the knee or in vertebrae. The joint spacer may be used as an intervening layer as needed, such as when an individual is awaiting knee or back surgery. For example, if cartilage is degraded, the filler may be used in its place. Further, if a meniscus that caps a joint is damaged or degraded, the filler may be used as a replacement. The filler can be considered an artificial disc, when vertebrae are damaged or degraded. The advantage of the filler in his use is that it is injectable, moldable, and ultimately removable. Thus, if an individual is awaiting surgery, such as knee replacement surgery, the filler can be injected in a minimally invasive manner and removed once the replacement joint is ready, or the surgery is complete.

As a tissue spacer, the filler can be used in a manner which is generally similar to the above-noted joint spacer. However, the tissues to be separated need not be joints, but any tissues requiring spaced proximity to each other can be separated with the filler. The implant can be used in a similar manner to fill a cavity. In a region of the body where tissue has been removed, the implant may be inserted in order to conserve the normal appearance of that tissue, or to protect the underlying area. As an example of this, injury or trauma to the eye may benefit from use of the filler. In such instances in which the filler is used as a tissue spacer, the implant can also be removed in stages or re-shaped, so that it is not all removed at the same time if the spacing requirements of the tissue change over time.

For breast augmentation or reconstruction, the thermally reversible filler can be used as an alternative to silicone or saline as fillers of breast implant, and advantageously can achieve a high viscosity once the gel is thermally formed in a semi-solid state. The shape and size of the breast implant can be varied by exploring the thermal reversibility of the filler. Augmentation or reconstruction of other body areas also falls within the scope of the invention.

The thermally reversible implant or filler of the invention can be used as a temporary sealant in surgical procedures, for example as an option to severing or cauterizing blood vessels. A blood vessel may be sealed by injection or insertion of the implant within the lumen of the vessel or by covering an area of bleeding tissue.

The thermally reversible filler can be used to block blood flow. For example, to seal the blood flow feeding a tumor, injection of the implant in liquid form into that vessel can be affected. This effect would be reversible through cooling. The invention can be applied for any number of surgical applications in which it is it is desirable to restrict or redirect blood flow, advantageously in a reversible way.

In instances where damage has been done to certain structural components of the body, the implant may be used as support for that organ or tissue, or as a bulking agent or tissue expander to provide structural integrity to the tissue or surrounding area. For example, if there is damage to a biological conduit, such as the uretor, or a sphincter, such as of the bladder, the implant may be used to alter the shape or to surround that particular tissue to help it maintain the desired shape required for proper function. This may be done by inserting the implant into the tissue of interest or by forming an implant to surround or abut the tissue of interest to achieve the required outcome.

Further, the implant can be used for reversible birth control applications in both women and men. For example, in men the implant may be used for implantation with the vas deferens to cause blockage thereof. This blockage can be reversed by cooling the area to a temperature below which the implant becomes liquid, so that the blockage can be removed. In women, the implant can be applied or implanted as a cervical sealant so as to prevent conception. By cooling the area of application to a temperature below which the implant becomes liquid, the sealant is removed. In both cases, only minorly invasive methods are required for both application and removal of the implant.

The invention relates to a method of forming a removable implant in an animal comprising inserting a thermal reversible gel into said animal, said gel having a semi-solid gel form at body temperature and a liquid form upon cooling to a temperature below a threshold temperature. The threshold temperature may differ depending on the nature of the gel or polymer used and the intended location in the body of the implant or filler. The threshold temperature is preferably less than body temperature at the site of implantation, more preferably at least 5° C. less than body temperature. Ideally, The threshold temperature is 5 to 15° C. below body temperature; in this way, cooling need only be applied locally to achieve the appropriate temperature differential to cause liquefaction of the gel or polymer.

Once the temperature of the gel form which the implant is formed is below the threshold temperature, it is liquefied, re-shapable, or removable.

Removal is then affected by any acceptable means such as through aspiration, washing or dabbing the liquid from the area. Removal of the implant can be effected implant by cooling the body in the region of the implant to a temperature below the threshold temperature and extracting the implant.

Also, the implant can be re-shaped by using the step of cooling the body in the region of the implant below the threshold temperature, re-shaping or re-sizing the implant in the liquid state and then forming a solid gel again of the new shape and volume.

The invention also relates to a method of forming the implant in situ or in vitro. The gelable composition is convertible from liquid to gel. Thus, the implant would be formed by inserting the composition into a subject at a temperature below the gelation temperature. The composition would be heated by the body or an external source to a temperature above the gelation temperature to form an implant in situ. Alternatively, the step of heating the composition to at least the gelling temperature can be used to form the implant in vitro, prior to implantation in the body.

The polymer A(B)ₙ in accordance with an aspect of this invention undergoes gel formation in response to temperature changes. This results from temperature-sensitive aggregation of the arms (B) of the copolymer. Thus, at the temperature that the arms (B) aggregate, gelation of the ABn copolymer occurs. It is this aggregation of the arms that physically (as opposed to chemically) cross-links the ABn copolymers to each other to form a gel. The network structure does not rely on micelle formation. In the resulting gel, the copolymer incorporates an equilibrium quantity of solvent due to the compatibility between core A and the solvent, thereby forming a solvent-containing gel.

As a result the gel that is formed is a strong gel with little syneresis, in contrast to gels which rely on micelle formations. A measurement of the strength of a gel is the breaking strength. Increasing breaking strength must be balanced with low syneresis for each application, and thus, the preferred breaking strengths will vary as a function of the desired application. Examples of breaking strengths in accordance with the invention are greater than 200 Pa, more preferably 500-1000 Pa.

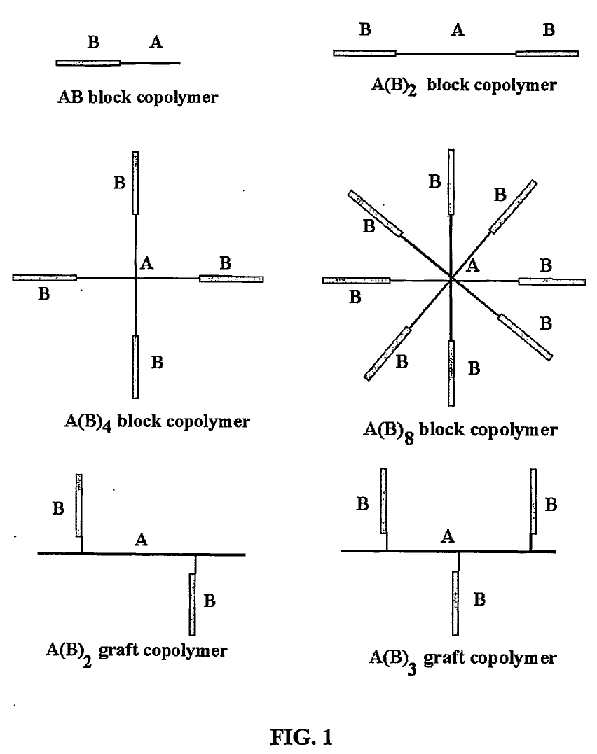

The copolymer contains an unresponsive core (A) to which a varying number of temperature-responsive arms (B) are attached. Thus, the copolymer has a general structure A(B)ₙ. The arms (B) can be attached at any point along the core (A), provided the arms are accessible to the arms of other molecules for intermolecular aggregation upon changes in temperature. For example, the arms may be attached to the ends of the core, thus forming a block or star copolymer, or may be attached along the chain of the core, thus forming a graft copolymer. FIG. 1 diagrammatically illustrates one-arm, two-arm, four-arm and eight-arm block copolymer structures A(B)₂, A(B)₄ and A(B)₈, and graft copolymer structures A(B)₂, A(B)₃, with comparison to block structure AB.

The Core. The core (A) may be a homopolymer, or the core (A) may itself be a copolymer (random, block or graft), either linear or branched, provided that A is soluble over the temperature range of interest.

Core (A) may either be provided as a stable compound or as a degradable compound. In the case where the core is degradable, the copolymer or copolymer composition degrades over time under appropriate conditions. For example, if the core is biodegradable in a physiological system, eventually the polymer structure will break down, resulting in release of the arms, and ultimately removal of the copolymer structure from the physiological system.

A number of possible cores (A) can be used according to the invention. The core may be selected from any synthetic, natural or biological polymers, including but not limited to polyethylene glycol (PEG) of varying molecular weights and degrees of branching, polyvinyl pyrrolidone, polyvinyl alcohol, polyhydroxyethylmetacrylate, and hyaluronic acid. Optionally, the core can have reactive groups at a variety of positions along or within its structure.

The Arms. The arms (B) are chosen such that B itself would switch between being soluble and insoluble in the selected solvent in the temperature range of interest.

A number of choices for the arms (B) of the copolymer exist, including, but not limited to poly-N-isopropyl acrylamide (PNIPAAm), which is a temperature responsive polymer. Other temperature-responsive polymers for use as B include hydroxypropylmethyl cellulose and other methyl cellulose derivatives, poly(ethylene glycol vinyl ether-co-butyl vinyl ether), polymers of N-alky acrylamide derivatives, poly(amino acid)s or peptide sequences such as silk and elastin peptides. poly(memthacryloy L-amine methyl ester), poly(methacryloy L-alanine ethyl ester). Nitrocellulose may be used as arms (B), for example when ethanol is used as solvent. Nitrocellulose in ethanol is known to form gel upon warming (Newman et al., J. Phys. Chem. 60:648-656, 1955). In the selection of arms (B), one of skill in the art would also consider whether the selected arms allow formation of a copolymer with the desired properties, which could easily be determined by observing the properties.

Arms (B) may be formed from a copolymer, for example a copolymer of vinyl ether of ethylene glycol and butyl vinyl ether, which may be used in an aqueous solvent system. For a copolymer, the LCST (lower critical solution transition) beyond which a polymer changes solubility, depends on the mole ratio of the constituent components. In the examples given by Kudaibergenov et al. (Macromol. Rapid. Commun, 16: 855-860, 1995). The LCST values range from 20° C. to 90° C. over a mole ratio range of 72.28 to 95:5.

Arms (B) may be formed from poly(methacryloyl-DL-alanine methyl ester) or derivatives thereof. In the paper by Ding et al. (Phys. Chem., 42 (4-6): 959-962, 1993), the LCST of the examples given are between 20° C. To 40° C. The gel swells at low temperature (i.e., 0° C.) and starts to de-swell upon warming to 20° C. or above.

Further, the arms (B) may be formed of methyl cellulose or derivatives thereof. Depending on specifics of the chemical composition, especially the degree of methylation, methyl cellulose and its derivatives were report to have a LCST in the range of 40° C. to 70° C. (Nishimura et al., Macromol. Symp., 120: 303-313, 1997).

The range of interest in which B converts from soluble to insoluble in the solvent of choice, independently of A, as preferably between 5 to 50° C., more preferably from 20 to 35° C.

The arms (B) may be attached to the unresponsive core (A) at any location on the core, as long as the arms remain accessible to the arms of adjacent copolymer molecules, as part of the composition comprising the ABn copolymer and the solvent. This structure allows for intermolecular aggregation of arms (B) when temperature is altered such that the B component of ABn would become insoluble in the selected solvent. For example, arms B may be positioned at the end of the core, thus forming a block copolymer (including star-shaped copolymers), or along the chain of the core thus forming graft copolymers.

As used herein, the structure “A(B)n” denotes a copolymer having arms (B) positioned on the core (A) in any manner, so as to form a block or graft copolymer. Arms (B) may be located at one or more ends of A, forming a block or star copolymer configuration, or may be located along the length of the core, thereby forming a graft copolymer, with B positioned as “brushes” along the core, or may be positioned randomly along the core, provided the arms are accessible for aggregation with the arms of adjacent molecules.

Further, as the structure “A(B)n” is understood to mean that A and B are present in the specified ratio within a given molecule, but that the covalent bond between A and B may also comprise an additional component, resulting in A and B being covalently linked through such an additional component. An example wherein the additional component is a reactive spacer is described in more detail below.

For any given copolymer molecule, n is a integer greater than 0, preferably greater than 1, and may be 2, 3, 4, 5, 6, 7, or 8, for example. Thus, for example, when n is 2, such that the copolymer is represented by AB₂, the ratio of arms to core in the architecture of the copolymer molecule is 2:1. For example, the ratio of arms to core can be 4:1 (n=4) or 8:1 (n=8). The number of arms is not limited, provided that core is of adequate size to accommodate the selected number of arms, while still allowing the arms of one copolymer molecule to access the arms of an adjacent copolymer molecule when in solution. The selection of the number of arms may also depend on the desired properties of the gel, for example, to achieve a stronger or weaker gel the number of arms may be adjusted.

The relative concentration of A to B will depend upon the application. In one aspect, the concentration of A is 1 to 50 mol %, 5 to 35 mol %, or 5 to 25 mol %. In this case describing the relative mol %, A refers to the units comprising A and B refers to the units comprising B.

The gelable composition according to the invention may contain mixtures of A(B)n copolymers that contain different A components, different B components, or have different n, or any combination thereof. In this way, mixtures can be used to optimize gelation kinetics or to achieve gel properties desirable for a particular application. Thus, the gelable composition formed according to the invention may be comprised of a plurality of different copolymers. Taking into account the proportions of different copolymer architectures within the composition, an average A(B)n can be determined for the composition. In this case, the average n (n_{avg}) must be greater than 1; non-integer values of n_{avg} are possible for any particular gelable composition. For example if the composition contains a mixture of copolymers of varying architectures, such as 50% copolymer AB and 50% copolymer A(B)₂, the n_{avg} of the composition is 1.5. in the inventive composition, n_{avg}>1, taking into account all forms of A(B)n copolymers in the composition. For any individual copolymer molecule within the composition, n is an integer number, as described above. In compositions which contain a mixture of copolymers, it is possible to have a gel-forming composition comprising some copolymer molecules with n=1, some with n=4, etc. In order for such a composition to be gelable according to he invention, n_{avg}, should be adequately greater than 1, so that enough copolymer molecules with n>1 are present in the composition to allow formation of the gel network. In this way, copolymer molecules having the structure AB(n=1), which would not ordinarily form a gel with other AB copolymers, can become part of the gel network by having their single arm segment incorporated into the aggregates formed by the molecules having n>1. n_{avg} may be greater than 1.1, 1.2, 1.3, 1.4, 1.5, 1.6, 1.7, 1.8, 1.9, 2, 2.1, 2.2, 2.3, or 2.4, for example.

According to one embodiment of the invention, PEG is used as core A, poly(N-isopropyl acrylamide) (PNIPAAm), a temperature responsive polymer, is used for arms B. Copolymers are formed with varying numbers of PNIPAAm arms. These copolymers are water soluble at room temperature, forming low viscosity liquid aqueous solutions. However, upon heating, the copolymers rapidly and reversibly form strong gels (in less than a minute), exhibiting little syneresis.

Reactive Spacer. Reactive spacers “C” may be present between core A and arms B, thereby forming a copolymer of the generic structure A(CB)n. It is understood that A(CB)n is a variant or embodiment of A(B)n, as the structure A(B)n is understood to mean that A and B must he present in the specified ratio, but that the covalent bond between A and B may also comprise an additional component, resulting in A and B being covalently linked through component C.

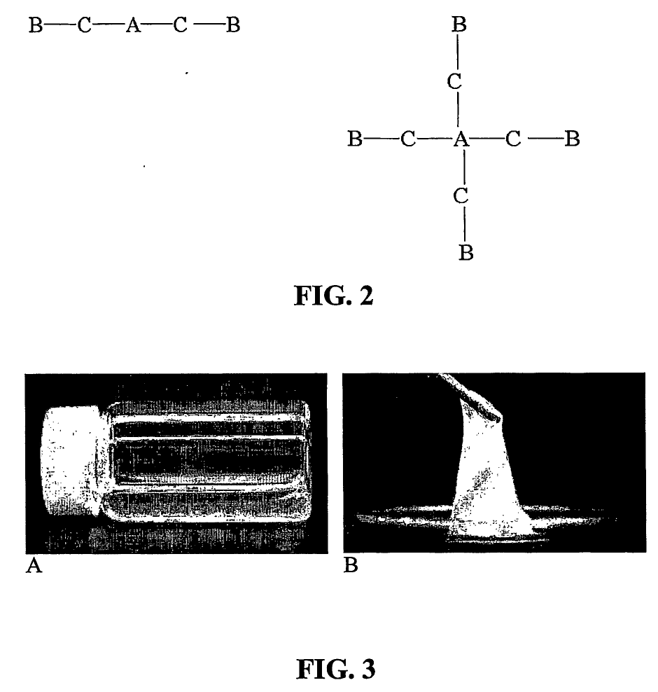

FIG. 2 illustrates two-arm and four-arm copolymer structures with reactive spacers C. As can be seen in FIG. 2, when a reactive spacer C is present between A and B, the basic structure of A(B)n is met, and merely includes an additional component C within the covalent bonds binding A to B. In the embodiment of A(CB)n, two covalent bonds bind A To B, specifically, the bond between A and C, and the bond between C and B.

Reactive spacers C may be incorporated to allow cleavage of the copolymer, for such purposes as for rendering the copolymer degradable under desired conditions. Reactive spacer C may degrade via any suitable reaction, including but not limited to chemical reactions, biochemical reactions, enzymatic degradation, or photo-induced reactions. In the case where a reaction of the reactive spacers results in cleavage of the copolymer, as C degrades, A(CB)n is spilt into individual A and B components. In the context of a physiological application, if core A and arms B are of low enough molecular weight, they can be cleared from the site and removed from the body via renal clearance.

Biologically Active Molecules. A biologically active molecule may be included in the invention either through covalent attachment of the molecule to the structure of the copolymer or by including the molecule in a copolymer composition. In the case where the biologically active molecule is included in the copolymer composition, but not incorporated into the copolymer itself, the biologically active molecule is optimally selected from those having some degree of solubility in the desired solvent.

According to an embodiment wherein the biologically active molecule D is attached to the copolymer, it may be bound to either the core (A) or the arms (B) in such a way that the attachment allows release of the biologically active molecule D from the copolymer. For example, a covalent attachment of D to A may occur via a degradable spacer, such as C, described above.

As with the introduction of reactive spacer (C) in the copolymer, introduction of biologically active molecule D, with or without spacer C, is considered an embodiment of A(B)n. It is understood that D maybe covalently attached to either A or B, and a copolymer polymer so formed would meet the requirement structure of A(B)n. The structure A(B)n is understood to mean that A and B must be present in the specified ratio, but that the covalent bond between A and B may also comprise an additional component such as D, through which the covalent attachment of A and B, may be directly achieved

According to a further embodiment of the invention, biologically active components may be included in me polymeric composition formed according to the inventions but without any covalent link to the polymer itself. Advantageously, when a gel is formed, a biologically active compound present in the polymeric solution becomes trapped in the gel structure. This arrangement is conducive to slow release of the biologically active molecule from the gel structure with a physiological environment.

A biologically active molecule for incorporation into the copolymer or copolymer composition may be any which causes a physiological change or effect, such as a low molecular weight compound, drug, antibody, growth factor, peptide, oligonucleotide, genetic sequence, or compounds that modulate cell behaviours such as adhesion, proliferation or metabolism. A biologically active molecule may be attached to the copolymer or included in the copolymer composition in order to promote the viability or proliferation of cells encapsulated in such gels, or to influence the production of compounds by such cells.

The Solvent. Various solvents may be used with the copolymer composition. The solvent may be aqueous, including water, sodium chloride solutions such as physiological saline, cell culture media, or any medium that approximates a biological system, such as extracellular matrix. Non-aqueous solvents may be used, or combination solvents including a polar organic and an aqueous component. For example, an alcohol may be used as the solvent, with or without water. Ethanol, methanol, isopropyl alcohol and other alcohols my be used as a solvent. Other polar organic solvents may be used alone or in combination with water. Non-polar organic solvents may be used with appropriate copolymers, such that A is soluble in the solvent, and B is convertible between soluble and insoluble as a function of temperature.

The term “solvent” may also refer to any prepared mixture of components which may include proteins, growth factors, buffers, ions, and other co-solutes, as well as solid particles.

For example, culture media and extra cellular solutions contain water in combination with a number of co-solutes which are considered part of the solvent. As described further in Example 6, varying the concentration of the buffer and/or other ions, thus changing the osmolarity, can be used to modify the gelation temperature of the copolymer in the solvent.

Further, other soluble components or additives, such as polymers may be included in the solvent. Such polymers may, for example, be synthetic polymers or copolymers that do not aggregate with the copolymer having A(B)n architecture. The solvent may contain, for example, the polymer used as core component (A) in the copolymer A(B)n. When such a polymer or copolymer is included in the solvent, it would not be considered in the calculation of n_{avg} unless it had a structure A(B)n and was capable of aggregation with arms B of the inventive copolymer. As an example of solvents which include polymers, PEG homopolymer, carboxymethylcellulose, and others may be included in the solvent. Other examples include sugars (sucrose, lactose, dextran), sugar alcohols, water soluble synthetic polymers (like poly vinylpyrrolodinone, and poly methacrylic acid), and starches. The use of additives can be employed to modify gel hydration/syneresis.

In addition, the solvent may contain solid particles for use in strengthening the gel composition.

The copolymer can be present in the solvent at any concentration that allows gelation to occur, for example a level of from about 5% to about 50% by weight, or from about 10% to about 25% by weight. This concentration depends on the nature of the solvent and the copolymer.

Modification for Implant and Filler Applications

The use of the copolymer for the applications described herein requires specific modification of gelation temperature, viscosity of the copolymer in solution below the gelation temperature and the physical properties (i.e., syneresis, breaking strength, elastic modulus and viscous modulus) of the gel above the gelation temperature. For most implant and filler applications, it is desirable to deliver the copolymer as non-invasively as possible (e.g., by injection through needles or catheters); therefore, liquid viscosities of less than 10,000 cP are preferred, more preferably less than 5000 cP, and less than 1000 cP most preferred. Gelation temperature may require modification depending on the temperature of the site of application (e.g., wrinkle filling requires a lower gelation temperature because skin is cooler than body temperature) or on the balance of gelation kinetics versus delivery time. For instance, use of the copolymer to block blood flow would require rapid gelation upon delivery. For most filler applications, it is desirable for the solid gel to retain the same volume that was delivered. Thus, copolymers exhibiting syneresis values less than 40% are desirable, values of less than 20% are preferred, less than 10% are more preferred, and less than 5% are most preferred. For filler applications, it may be important that the solid gel rheological properties are compatible with the surrounding tissue. For example, hyaluronic acid-based commercial wrinkle fillers have elastic and viscous moduli of approximately 100-300 Pa and 50-150 Pa, respectively. Other applications may require that the solid gel resist specific applied forces (e.g., blood flow or joint compression). For certain applications, a breakup strength of more than 200 Pa, preferably more than 500 Pa is desirable. The examples that are included demonstrate how the liquid viscosity, gelation temperature and the physical properties of the solid gel can be modulated by changing copolymer concentration in the solvent, copolymer composition, copolymer structure, and the incorporation of various additives (e.g., ions, macromolecules and solid particles) into the copolymer solution. These modifications enable a wide range of filler and implant applications.

Additional Applications of Invention. The invention may be used as described above, or as described herein below. Physiological and clinical applications of the invention include, but are not limited to, delivery of biologically active molecules, tissue and biomedical engineering, and therapeutics.

The invention can be applied to delivery of biologically active molecules, for example but not limited to in vitro formation of drug delivery systems, in situ drug delivery, in situ gene delivery. The inventive polymer may be used to form drug delivery systems in vitro, which could then be implanted into a physiological region of a subject. Drug delivery systems may be formed in situ by suspending drug-containing particles in the copolymer composition, then injecting the composition into, or applying the composition onto specified sites of a subject causing gel formation to occur in vivo. Genes may be delivered in vivo using the inventive polymers and compositions. Gene delivery systems in situ can be formed by suspending gene-containing vesicles in the polymer solutions, then injecting the solutions into, or applying the solutions onto specified sites of patients causing gel formation to occur in vivo. Possible sites for implantation for in vitro formed systems or for insertion of in situ forming systems of biologically active molecules include but are not limited to periodontal cavities, intramuscular sites, subcutaneous sites, tumors, bones, joints, intramuscular sites, sites that have been exposed by surgery, and wound sites.

Further, the invention may be used for an vitro or in situ encapsulation of cells. For encapsulation of cells in vitro, cells can be grown in incubation medium to which the copolymer is added when desirable, so as to keep cells in suspension at certain temperatures, but to retain them in a gel when the temperature is changed. Encapsulation of cells may also occur in situ by suspending cells in the copolymer composition under conditions at which the composition is a liquid (for example, below LCST), then injecting the composition into, or applying the composition onto specified sites of patients causing gel formation to occur in vivo. The sites for in situ injection of suspended cells in the composition, or for insertion of an in vitro formed implant of encapsulated cells can be selected from, but are not limited to, periodontal cavities, intramuscular sites, subcutaneous sites, tumors, bones, joints, intraocular sites, sites that have been exposed by surgery, and wound sites.

For applications involving encapsulated cells, the length of chain segments between the physical crosslinks of the copolymer may be selected such that the mesh size between crosslinks provides the appropriate molecular weight cut-off to provide immunoisolation of the encapsulated cells for the intended host while allowing the diffusion of desired nutrients to the cell, and the release of desired agents from the encapsulated cells to the host. In an application of in situ forming cell-containing gels, the copolymer would be soluble in water at ambient conditions (i.e. room temperature), and the composition including suspended cells is injected into or applied onto a patient at the desired site. Body temperature triggers gel formation, thus causing the cells to be trapped in the gel at the site of injection or application cell proliferation and secretion of desired substances from the cell may then occur.

In cell-containing applications, it may be particularly advantageous to incorporate into the gel peptides or growth factors that promote cell adhesion, cell proliferation or otherwise influence cell metabolism in the desired manner. Such compounds may either be covalently linked to the copolymer, or incorporated in solid particles or liquid droplets that are co-encapsulated in the composition with the cells.

### EXAMPLES

Examples of the invention are presented below to illustrate the invention, but not to limit the scope of the invention.

### Example 1

### Synthesis of Thermoreversible Gel (TRG)

An example of TRG synthesis conditions is as follows. Polyethylene glycol (PEG, 2.42 g), N-isopropyl acrylamide (NiPAAm, 1.75 g) and degassed endotoxin-free distilled water (44 ml) were measured and transferred to a 100 mL glass, round-bottom reaction flask. The reactor was flushed with nitrogen gas and placed in a 50° C. water bath for at least 15 minutes. A ceric ammonium nitrate solution (0.6370 g in 6 ml 1M HNO₃) was then added to the reactor via syringe. The reaction proceeded for 3 hr after the addition of the cerium solution. After 3 hr, 50 mL of degassed endotoxin-free water 4° C. was added to the reactor and the reaction vessel was placed in an ice bath for ˜15 minutes to dissolve the synthesized TRG.

The increased reaction temperature (50° C. from 30° C.) and the addition of nitric acid were adopted to increase cerium initiation activity and polymerization rate allowing for reduced reaction times (3 hr from 24 hr). In addition, the amount of ceric salt added was also reduced (5.5 fold) making removal of residual cerium contamination from the synthesized gel simpler.

### Example 2

### TRG Purification

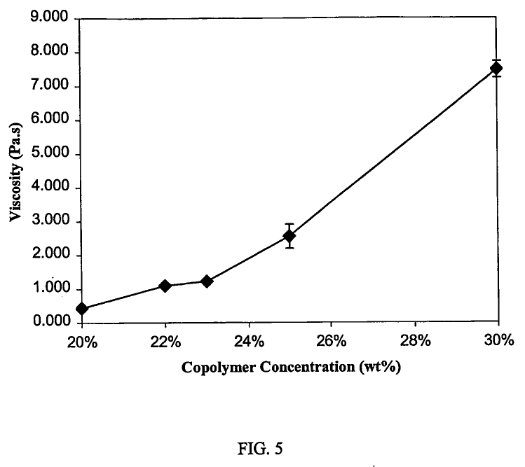

Precipitation of cerium salts resulting from the addition of sodium bicarbonate at the end of the reaction was followed by a two-step filtration procedure. First, the solution was vacuum filtered using a filter aid (Celpure™, Aldrich) and then vacuum filtered a second time using a 0.02 μm membrane. The filtered solution was then freeze-dried and the resulting solid was extracted in warm water (50-60° C.) at low concentration (5-10% w/v) for 24 h to remove water-soluble extractables (primarily unreacted PEG). The solid, swollen TRG was then filtered and rinsed with warm water. The extractions may be repeated as many times as necessary to attain a constant TRG composition (as determined by NMR spectroscopy), normally 3-5 extractions. Finally, the extracted material was dissolved in distilled water at 5% wt and filtered through a 0.22 μm membrane and freeze-dried to remove any remaining fine cerium-containing impurities. In this way, the Applicant were able to reduce the residual cerium content of the dry gel from >500 ppm to less than 20 ppm. FIG. 4 shows the effective removal of impurities detected by gel permeation chromatography resulting from the filtration/extraction procedure. In addition, this simple, relatively fast and effective technique reduced purification time from 4 weeks to 2 weeks.

### Example 3

### Modification of TRG Composition

Modification of the synthesis and purification procedures resulted in alteration in TRG composition (i.e. increased PEG content). Table 1 illustrates the effect of varying gel PEG content on material properties. As the PEG content of the TRG is increased from 6 to 17 mol %, the resulting gel becomes softer due to decreasing NiPAAm effective crosslink density. In addition, the room temperature viscosity of the TRG solution decreases with increasing PEG content. The gelation temperature is insensitive to alternation in PEG content. Therefore the increased PEG content resulting from modification to the synthesis and purification procedures yields a material that is significantly easier to inject (due to its reduced viscosity) but softer (lower G′). The high PEG content solid gel at 20% (w/w) is injectable through high gauge (27 and 30) needles and similar in stiffness to commercially available wrinkle filler materials (e.g. Hyalform and Restylane), making this formulation particularly useful in that application. Other applications may require different formulations. For example, the low PEG content TRG is not readily injectable (except through low gauge needles, e.g. 18) but may be strong and stiff enough for use as a spacer in applications where injection through large needles is acceptable.

TABLE 1
Effect of TRG PEG content on material properties.

| PEG content | Gel Temp. | Viscosity | G′ | G″ | |
|---|---|---|---|---|---|
| (mol %) | (° C) | (cP) | (Pa) | (Pa) | δ |
| 6 | 32.9 | 1,500-15000 | 3000-5,000 | 1800-3,000 | 0.6 |
| 12 | 32.3 | 1,100-1,500 | 155-225 | 60-90 | 0.25-0.55 |
| 17 | 32.7 | 250-350 | 135-215 | 110-130 | 0.55-0.85 |

### Example 4

### Effect of Concentration an Solution Viscosity and Injectability

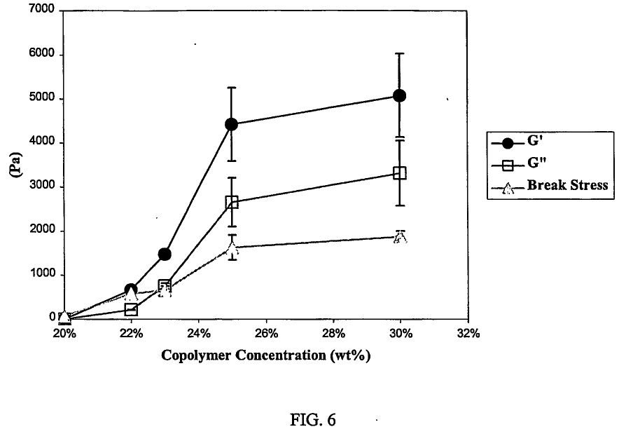

Copolymer solution viscosity (at 20° C.) was found to increase non-linearly with increasing solution concentration (FIG. 5), ranging of from 0.4 to 7.5 Pa·s at 1000 s⁻¹ shear rate. For reference, molasses is considered to be a high viscosity fluid (5-10 Pa·s) and water (0.001 Pa.s at RT) is a low viscosity fluid. Experimentally, the Applicant found that solutions with viscosities greater than 2.5 Pa·s at room temperature (at 1000 s⁻¹ shear rate) were very difficult to inject through 30 and 27 gauge needles (needle size typically used for wrinkle filler injections).

### Example 5

### Effect of Concentration on Gel Rheological Properties

The concentration of the copolymer solution was varied from 20-30% w/w and rheological properties after gelation were measured in order to determine the minimum concentration that would deliver an acceptably strong gel for filler applications. The rheological parameters measured were elastic modulus (G′), viscous modulus (G″) and breaking stress. The elastic modulus is a measure of gel stiffness, while the viscous modulus quantifies the resistance to flow and the breaking stress indicates the cross-sectional force required to break the gel (gel strength).

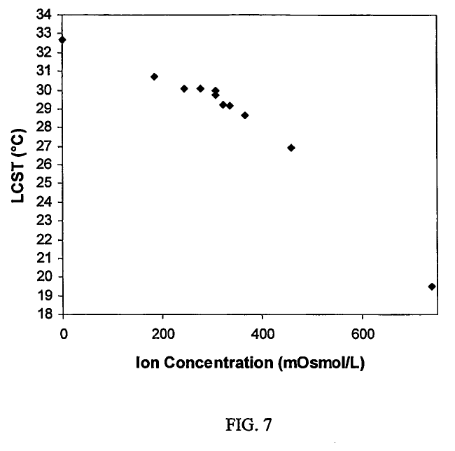

Elastic modulus (G′), loss modules (G″) and stress at break all increased with increasing copolymer concentration in the gel (FIG. 6). These results indicate that increasing copolymer solution concentration results in increasing gel strength and stiffness. Therefore, the Applicant are able to easily modulate the physical properties of the gel by simple alterations in solution concentration. In comparison, commercially available wrinkle-filler products based on modified hyaluronic acid (Hyalform® and Restylane®) exhibit G′ values on the order of 100 Pa and G″ values roughly one half to one third the G′ value. Therefore, the TRG may be formed into a similar or significantly stiffer gel than Hyalform® and Restylane® making it a potentially useful wrinkle filler and tissue filler in applications with widely varying mechanical requirements.

### Example 6

### Modification of Gelation Temperature by Changing Osmolarity

Since the temperature of gelation and dissolution was anticipated to effect the ease of delivery, reshaping and removal of the gel in tissue filler applications, the Applicant examined methods for easily tuning the gelation temperature. In particular, the effect of TRG solvent osmolarity on gelation was investigated. Water, saline and phosphate-buffered saline solutions were prepared to produce a range of osmolarities (0 to 740 mOsml/L) at 23 wt % and the gelation temperature was measured by differential scanning calorimetry. FIG. 7 shows the effect of solvent osmolarity on TRG gelation temperature. Increasing osmolarity resulted in decreasing gelation temperature, reducing the temperature from approximately 32.5° C. to 19.5° C., making it possible to broadly tune the gelation point easily.

### Example 7

### Modification of Gel Hydration by Incorporation of Additives to the Solvent

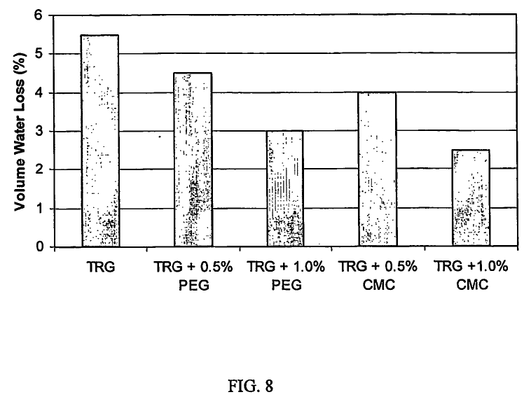

The importance of volume retention on gelation for tissue filling applications led us to examine methods to modify/minimize liquid loss (syneresis) on gelation. To this end, the Applicant investigated to effect of including hydrophilic additives into the TRG solutions on syneresis. TRG solutions were prepared at 20% (w/w) in distilled water and varying amounts of polyethylene glycol (PEG, mol wt.=1,000,000) and carboxymethylcellulose (CMC, low viscosity) were added. PEG and CMC were dissolved at 0.5 and 1.0% (w/v) into the original TRG solution to evaluate the impact of type and concentration of additive. One milliliter of each sample solution was placed in a 6 mL glass vial and placed in an oven at 37° C. for 24 hr. Then, the sample was removed from the oven and the volume of expelled solvent was measured and reported as a percentage of the original solution volume. FIG. 8 shows the results of the study. The TRG solution containing no additives exhibited relatively low syneresis (5.5%). Addition of both PEG and CMC resulted in a concentration-dependent reduction in gel syneresis (i.e. increasing additive concentration reduced syneresis) to as low as 2.5%. This effect is presented to occur due to an increase in the negative entropy of mixing for the TRG solution resulting from the ability of the PEG and CMC to structure water and represents a convenient method for tailoring gel volume retention.

### Example 8

### Biocompatibility/Safety Testing

Basic biocompatibility/safety testing was performed on 23% (w/w) TRG solutions that were sterilized by steam autoclave. Three tests were performed to evaluate biocompatibility: intracutaneous reactivity of gel extracts; in vitro biological reactivity of gel extracts and dermal sensitization for the gel. The gel extracts showed negligible response in the intracutaneous reactivity test and therefore the material was deemed to meet the requirements of the test criteria for biological responses for intracutaneous reactivity. The gel extracts also showed no reactivity at 0.2 g/mL extraction ratio (in cell culture medium) for L-929 fibroblast cells in the in vitro biological reactivity elation test. Finally, no dermal sensitization or irritation was detected when the gel was directly applied. Therefore, the material passed all of the biocompatibility/safety tests performed.

### Example 9

### Stability of TRG

Stability studies on the TRG were performed using temperature-accelerated aging conditions to determine shelf-life. Rheological properties, gel transition temperature and molecular weight were measured after storage under conditions (54° C.) that are equivalent to storage at 4° C. (the anticipated storage temperature) for 1 and 2 years. The data collected on material properties after temperature-accelerated storage indicates that there is little change in properties over storage time (Table 2). No significant change in molecular weight, elation temperature or solution viscosity was detected indicating that there was no measurable alteration in the TRG chemistry. The modulus values (G′ and G″) and breaking stress did increase with increasing storage time meaning that the solid gel became stiffer and stronger with time. Since none of the other material characteristics changed with time it is believed that a small amount of evaporative water loss with storage at the elevated temperature increased the gel physical strength. Importantly, there was no evidence of degradation or reduction of material properties during storage.

TABLE 2
Effect of accelerated aging on material properties of TRG.

| Storage | | | | Breaking | Gelation | Molecular |
|---|---|---|---|---|---|---|
| Time | Viscosity | G′ | G″ | Stress | Temp. | Weight |
| (years) | (Pa s) | (Pa) | (Pa) | (Pa) | (° C.) | (g/mol) |
| 0 | 0.95 | 2370 | 1590 | 1080 | 32.3 | 238200 |
| 1 | 1.15 | 3760 | 2770 | 1340 | 32.1 | 244700 |
| 2 | 0.90 | 4560 | 3550 | 1410 | 32.2 | 235000 |

### Example 10

### Effect of Autoclaving

The most desirable method for sterilization of the TRG is terminal steam autoclaving (i.e. autoclave steriliation of final TRG solution) at 120° C. for 30 minutes. It was thus necessary to determine the effect of autoclaving on TRG material properties (solution viscosity, solid gel rheology and gelation temperature). The solution viscosity and gelation temperature (T_{gel}) were not significantly affected by the sterilization process, but the elastic and viscous moduli increased after autoclaving (Table 3). Most likely, the slight change in the elastic and loss moduli resulted from minor water loss during autoclaving. As discussed above, the rheological properties of the gel are dependent on solution concentration.

TABLE 3
Effect of steam autoclave sterilization on TRG material properties.

| Property | Before Sterilization | After Sterilization |
|---|---|---|
| Viscosity (at 1000 1/s) | 0.97 ± 0.20 (Pa · s) | 0.96 ± 0.18 (Pa · s) |
| G′ | 422 ± 91 (Pa) | 810 ± 89 (Pa) |
| G″ | 168 ± 34 (Pa) | 430 ± 47 (Pa) |
| Stress at Break | 296 ± 14 (Pa) | 492 ± 14 (Pa) |
| Gelation Temperature (T_{gel}) | 32.2 ± 0.3 | 32.3 ± 0.4 |

The above-described embodiments of the present invention are intended to be examples only. Alterations, modifications and variations may be effected to the particular embodiments by those of skill in the art without departing from the scope of the invention, which is defined solely by the claims appended hereto.

1. A thermally reversible biological implant comprising a copolymer and an aqueous solvent, the copolymer having the structure A(B)n, wherein:
n is an integer greater than 0;
A is soluble in the solvent;
B is convertible from soluble to insoluble in the solvent as a function of temperature; and
the implant is convertible from liquid to gel between 5 and 37° C.

2. The implant according to claim 1, wherein:
A is selected from the group consisting of polyethylene glycol (PEG), polyvinyl pyrrolidone, polyvinyl alcohol, polyhydroxyethylmetacrylate, and hyaluronic acid; and
B comprises poly-N-isopropyl acrylamide (PNIPAAm).

3. The implant according to claim 1, wherein n is greater than 1.

4. The implant according to claim 1, wherein n is greater than 2.

5. The implant according to claim 1, wherein n is an integer from 3 to 8.

6. The implant according to claim 1, comprising at least two of the copolymers of structure ABn, wherein n_{avg} is greater than 1.

7. The implant of claim 1, wherein A is present in a concentration of 1 to 50 mol %, based on ABn, preferably 5 to 35 mol %, more preferably 5 to 25 mol %.

8. The implant according to claim 1, wherein the copolymer(s) is present in the solvent at a concentration of from 5% to 50% by weight.

9. The implant of claim 1, having a syneresis of less than 40%, preferably less than 20%, more preferably less than 10%, even more preferably 5% or less.

10. The implant of claim 1, having a breaking strength of more than 200 Pa, preferably 500 to 1000 Pa.

11. The implant of claim 1, wherein the solvent includes pharmaceutically acceptable hydrophilic polymers.

12. The implant of claim 1, wherein the solvent includes pharmaceutically acceptable ions.

13. The implant of claim 1, wherein the solvent includes solid particles.

14. The implant of claim 1, having a viscosity of less than 10000 cP, preferably less than 5000, more preferably less than 1000 cP.

15. (canceled)

16. A method for blocking the vas deferens comprising implanting the implant of claim 1 within the vas deferens.

17. A method for sealing the cervix comprising implanting the implant of claim 1 on the cervix.

18. A method of forming a removable implant according to claim 1 in an animal, comprising the steps of (i) forming a gelable composition comprising the copolymer and the solvent, and (ii) inserting said composition into a subject to form an in situ implant or heating said composition to at least said gelling temperature to form an in vitro implant.

19. The method of claim 18, wherein the composition is liquefied, and thus re-shapable, re-sizable, or removable at a temperature below a threshold temperature.

20. The method of claim 18, additionally comprising the step of removing the implant by cooling the body in the region of the implant to a temperature below the threshold temperature and extracting the implant.

21. The method of claim 18, wherein the osmolarity of the composition is varied to modify the gelation temperation.

22. The method of claim 18, wherein the solvent includes hydrophilic additives to modify the syneresis of the implant.

23. The method of claim 18, wherein the solvent includes solid particles to modify the strength of the implant.

24. A process for preparing a thermally reversible gel by reacting PEG and NiPAAm in in the presence of ceric ammonium nitrate.

25. The process according to claim 24, additionally comprising removing cerium to form a low cerium gel.

26. The process of claim 23, further comprising purifying the gel by extraction.

27. (canceled)

28. (canceled)

29. The method of claim 18, wherein the implant is a wrinkle filler, a joint spacer, a tissue spacer, a tissue expander, a vessel blocker, a cosmetic enhancer, or a breast implant filler.
